(19) United States
(12) Patent Application Publication
(10) Pub. No.: US 2010/0125204 A1
(43) Pub. Date: May 20, 2010

(54) Ultrasound System And Method Of Forming Three-Dimensional Ultrasound Images

(76) Inventors: Jae Heung Yoo, Seoul (KR); Sung Yoon Kim, Seoul (KR)

Correspondence Address: JONES DAY, 222 EAST 41ST ST, NEW YORK, NY, 10017, US

(21) Appl. No.: 12/622,253

(22) Filed: Nov. 19, 2009

(30) Foreign Application Priority Data
Nov. 19, 2008 (KR) 10-2008-0115423

(51) Int. Cl. A61B 8/14; G06K 9/00

(52) U.S. Cl. 600/443; 382/131

(57) ABSTRACT

The present invention relates to an ultrasound system and a method of forming three-dimensional ultrasound images of a plurality of region of interests (ROIs). The ultrasound system comprises: a user interface configured to receive reference plane selecting information for selecting a reference plane and region of interest (ROI) setting information for setting a plurality of ROIs on the reference plane image; an ultrasound data acquisition unit configured to transmit ultrasound signals to a target object and receive ultrasound echo signals reflected from the target object, the ultrasound data acquisition unit being configured to acquire three-dimensional ultrasound data corresponding to the plurality of ROIs based on the ultrasound echo signals; and a processor configured to form a plurality of three-dimensional ultrasound images corresponding to the plurality of ROIs based on the three-dimensional ultrasound data.

The present application claims priority from Korean Patent Application No. 10-2008-0115423 filed on Nov. 19, 2008, the entire subject matter of which is incorporated herein by reference.

### TECHNICAL FIELD

The present invention generally relates to ultrasound systems, and more particularly to an ultrasound system and a method of setting a plurality of regions of interest to form a plurality of three-dimensional ultrasound images.

### BACKGROUND

The ultrasound system has become an important and popular diagnostic tool due to its non-invasive and non-destructive nature. Modern high-performance ultrasound imaging diagnostic systems and techniques are commonly used to produce two- or three-dimensional images of internal features of patients (target objects).

Generally, the ultrasound system may provide a three-dimensional ultrasound image including clinical information such as spatial information and anatomical figures of target objects, which cannot be provided by a two-dimensional ultrasound image. The ultrasound system may transmit ultrasound signals to the target objects and receive ultrasound echo signals reflected from the target objects. The ultrasound system may form a reference plane image based on the ultrasound echo signals. The ultrasound system may form the three-dimensional ultrasound image corresponding to a region of interest (ROI) set on the reference plane image.

In the conventional ultrasound system, only one ROI is allowed to be set on the reference plane image. As such, only one three-dimensional ultrasound image corresponding to the ROI may be provided. Thus, there is a disadvantage in that a user of the ultrasound system may not be able to accurately diagnose the target objects.

### SUMMARY

An embodiment for forming three-dimensional ultrasound images is disclosed herein. In one embodiment, by way of non-limiting example, a system for forming three-dimensional ultrasound images, comprises: a user interface configured to receive reference plane selecting information for selecting a reference plane and region of interest (ROI) setting information for setting a plurality of ROIs on the reference plane image; an ultrasound data acquisition unit configured to transmit ultrasound signals to a target object and receive ultrasound echo signals reflected from the target object, the ultrasound data acquisition unit being configured to acquire three-dimensional ultrasound data corresponding to the plurality of ROIs based on the ultrasound echo signals; and a processor configured to form a plurality of three-dimensional ultrasound images corresponding to the plurality of ROIs based on the three-dimensional ultrasound data.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an ultrasound data acquisition unit.

FIG. 3 is a schematic diagram showing a reference plane image and ROIs.

FIG. 4 is a flow chart showing a method of forming three-dimensional ultrasound images of a plurality of ROIs.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

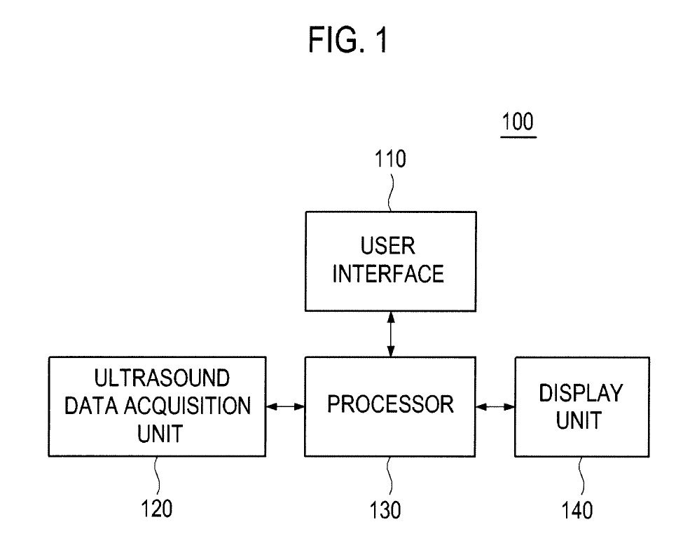

FIG. 1 is a block diagram showing an illustrative embodiment of an ultrasound system. The ultrasound system 100 may include a user interface 110, an ultrasound data acquisition unit 120, a processor 130 and a display unit 140.

The user interface 110 may allow a user to input reference plane selecting information for selecting one of reference planes and region of interest (ROI) setting information for setting a plurality of ROIs. The ROI setting information may include number, position and size of the plurality of the ROIs to be set on an image corresponding to the selected reference plane (hereinafter, referred to as “reference plane image”). The reference plane image may indicate a sectional image of a target object, which is vertical to an ultrasound probe (not shown). The user interface 110 may include at least one of a control panel (not shown), a mouse (not shown), a keyboard (not shown) and the like.

The ultrasound data acquisition unit 120 may transmit ultrasound signals to the target object and receive ultrasound echo signals reflected from the target object. The ultrasound data acquisition unit 120 may be further configured to form ultrasound data of the target object based on the received ultrasound echo signals.

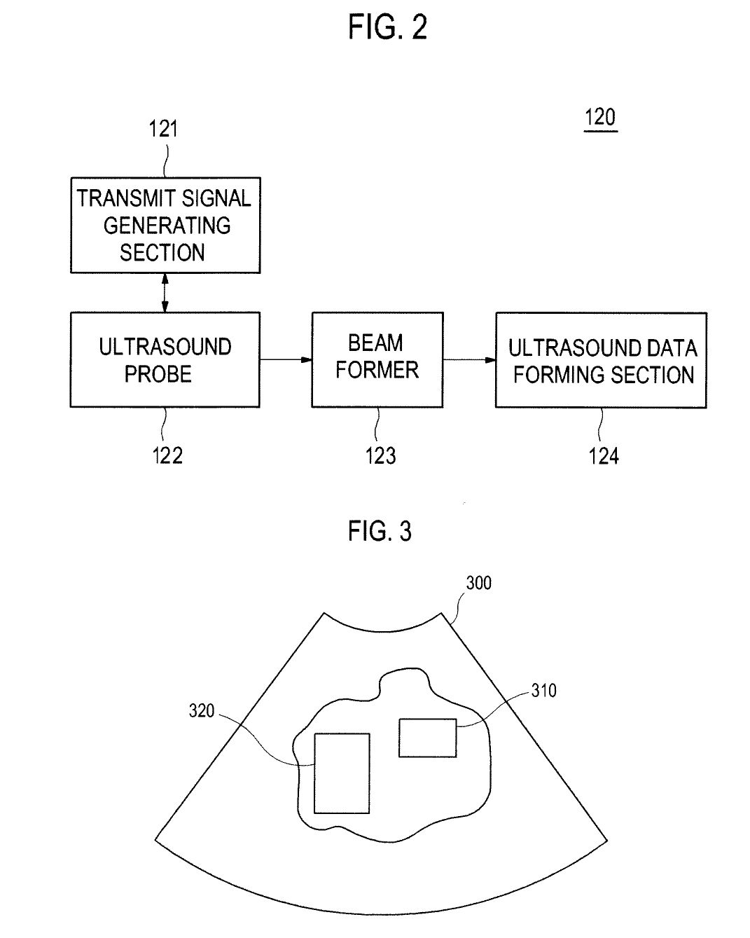

FIG. 2 is a block diagram showing the ultrasound data acquisition unit 120. The ultrasound data acquisition unit 120 may include a transmit (TX) signal generating section 121, an ultrasound probe 122 including a plurality of transducer elements (not shown), a beam former 123 and an ultrasound data forming section 124.

The TX signal generating section 121 may be operable to generate TX signals based on the reference plane selecting information. The ultrasound probe 122 may include an array transducer containing a plurality of elements. The TX signals based on the reference plane selecting information may be delayed in consideration of each of the transducer elements and focal points, and then applied to the transducer elements of the ultrasound probe 122. The TX signal generating section 121 may be operable to generate TX signals based on the ROI setting information. The TX signals based on the ROI setting information may be delayed in consideration of each of the transducer elements and focal points, and then applied to transducer elements of the ultrasound probe 122. FIG. 3 is a schematic diagram showing a reference plane image and ROIs. Assuming that the ROI setting information for setting first ROI 310 and second ROI 320 on the reference plane image 300 is inputted through the user interface 110, the TX signal generating section 121 may generate the TX signals for acquiring frames corresponding to the first and second ROIs 310, 320.

The ultrasound probe 122 may transmit the ultrasound signals in response to the TX signals and receive ultrasound echo signals reflected from the target object to thereby output receive signals. Assuming that the ROI setting information for setting first and second ROIs 310, 320 on the reference plane image 300 is inputted through the user interface 110, the receive signals may correspond to the first ROI 310 and the second ROI 320. The receive signals may be analog signals.

The beam former 123 may convert the receive signals, which are outputted from the ultrasound probe 122, into digital signals. The beam former 123 may be further configured to apply delays to the digital signals in consideration of the distances between the transducer elements and focal points to thereby output receive-focused signals. The receive-focused signals may correspond to the first ROI 310 and the second ROI 320.

The ultrasound data forming section 124 may form two-dimensional ultrasound data corresponding to the reference plane based on the receive-focused signals. Further, the ultrasound data forming section 124 may form three-dimensional ultrasound data corresponding to the plurality of ROIs based on the receive-focused signals.

Referring back to FIG. 1, the processor 130 may form the reference plane image based on the two-dimensional ultrasound data outputted from the ultrasound data acquisition unit 120 and a plurality of three-dimensional ultrasound images corresponding to the plurality of ROIs based on the three-dimensional ultrasound data outputted from the ultrasound data acquisition unit 120.

The display unit 140 may display the reference plane image and the plurality of three-dimensional ultrasound images corresponding to the plurality of the ROIs. The display unit 140 may include at least one of cathode ray tube (CRT) monitor, liquid crystal display (LCD) and the like.

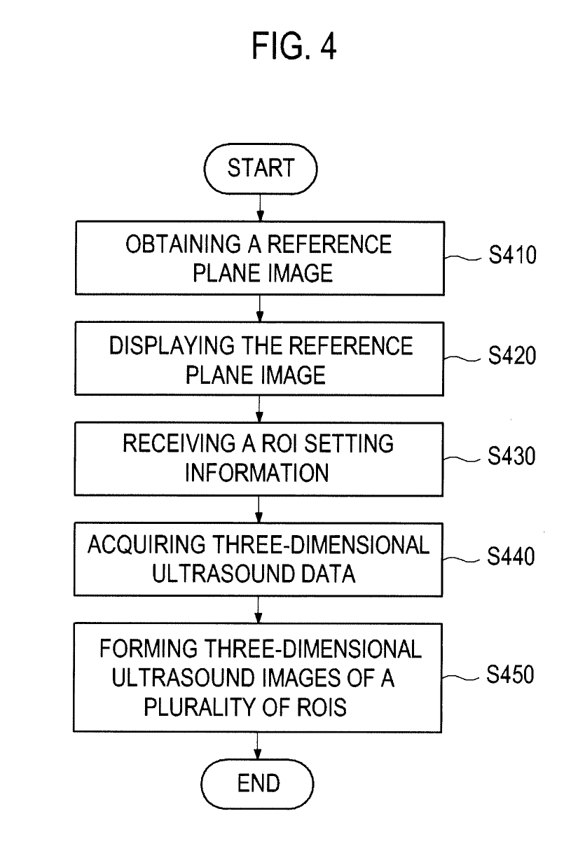

FIG. 4 is a flow chart showing a method of forming three-dimensional ultrasound images of the plurality of ROIs. In one embodiment, by way of non-limiting example, the ultrasound system 100 may obtain the reference plane image 300 (S410) and display the reference plane image 300 on the display unit (S420). Thereafter, the ultrasound system 100 may receive the ROI setting information from the user interface 110 (S430). The ultrasound data acquisition unit 120 within the ultrasound system 100 may acquire the three-dimensional ultrasound data corresponding to the ROI setting information (S440). The processor 130 within the ultrasound system 100 may form the ultrasound images of the plurality of ROIs (S450).

In another embodiment, the present invention may provide a computer readable medium, comprising instructions that, when executed by a processor performs a method of forming three-dimensional ultrasound images in the ultrasound system including the ultrasound probe, cause the processor to perform steps comprising: obtaining a reference plane image of a target object, displaying the reference plane image, receiving a region of interest (ROI) setting information on a plurality of ROIs set on the reference plane image, transmitting ultrasound signals to a target object and receiving ultrasound echo signals reflected from the target object to thereby acquire three-dimensional ultrasound data corresponding to the plurality of ROIs and forming the three-dimensional ultrasound images of the plurality of ROIs based on the three-dimensional ultrasound data. The computer readable medium may include a floppy disk, a hard disk, a memory, a digital versatile disc or the like.

Any reference in this specification to “one embodiment,” “an embodiment,” “example embodiment,” “illustrative embodiment,” etc. means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to affect such feature, structure or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a user interface configured to receive reference plane selecting information for selecting a reference plane and region of interest (ROI) setting information for setting a plurality of ROIs on the reference plane image;
an ultrasound data acquisition unit configured to transmit ultrasound signals to a target object and receive ultrasound echo signals reflected from the target object, the ultrasound data acquisition unit being configured to acquire three-dimensional ultrasound data corresponding to the plurality of ROIs based on the ultrasound echo signals; and
a processor configured to form a plurality of three-dimensional ultrasound images corresponding to the plurality of ROIs based on the three-dimensional ultrasound data.

2. The ultrasound system of claim 1, wherein the ROI setting information includes number, position and size of the plurality of ROIs.

3. The ultrasound system of claim 1, wherein the ultrasound data acquisition unit comprises:
a transmit (TX) signal generating section configured to generate TX signals based on the reference plane selecting information and the ROI setting information;
an ultrasound probe configured to transmit the ultrasound signals based on the TX signals to the target object and receive ultrasound echo signals reflected from the target object to thereby form receive signals;
a beam former configured to apply delays to the receive signals to thereby form receive-focused signals; and
an ultrasound data forming section configured to form two-dimensional ultrasound data corresponding to the reference plane and three-dimensional ultrasound data corresponding to the plurality of ROIs based on the receive-focused signals.

4. The ultrasound system of claim 1, further comprising:
a display unit configured to display the reference plane image and the plurality of three-dimensional ultrasound images corresponding to the plurality of the ROIs.

5. A method of forming a three-dimensional ultrasound image in an ultrasound system, comprising:
a) obtaining a reference plane image of a target object;
b) displaying the reference plane image;
c) receiving a region of interest (ROI) setting information on a plurality of ROIs set on the reference plane image;
d) transmitting ultrasound signals to a target object and receiving ultrasound echo signals reflected from the target object to thereby acquire three-dimensional ultrasound data corresponding to the plurality of ROIs; and
e) forming the three-dimensional ultrasound images of the plurality of ROIs based on the three-dimensional ultrasound data.

6. The method of claim 5, wherein the ROI setting information includes number, position and size of the ROIs.

7. The method of claim 5, wherein the step d) comprises:
d1) generating TX signals based on the ROI setting information;
d2) transmitting the ultrasound signals based on the TX signals to the target object and receiving ultrasound echo signals reflected from the target object to thereby form receive signals;
d3) applying delays to the receive signals to thereby form receive-focused signals; and
d4) forming three-dimensional ultrasound data corresponding to the plurality of ROIs based on the receive-focused signals.

8. The method of claim 5, further comprising:
f) displaying the plurality of three-dimensional ultrasound images corresponding to the plurality of the ROIs.

9. A computer readable medium comprising instructions that, when executed by a processor performs a method of forming three-dimensional ultrasound images in an ultrasound system including an ultrasound probe, cause the processor to perform steps comprising:
a) obtaining a reference plane image of a target object;
b) displaying the reference plane image;
c) receiving a region of interest (ROI) setting information on a plurality of ROIs set on the reference plane image;
d) transmitting ultrasound signals to a target object and receiving ultrasound echo signals reflected from the target object to thereby acquire three-dimensional ultrasound data corresponding to the plurality of ROIs; and
e) forming the three-dimensional ultrasound images of the plurality of ROIs based on the three-dimensional ultrasound data.

10. The computer readable medium of claim 9, wherein the ROI setting information includes number, position and size of the ROIs.

11. The computer readable medium of claim 9, wherein the step d) comprises:
d1) generating TX signals based on the ROI setting information;
d2) transmitting the ultrasound signals based on the TX signals to the target object and receiving ultrasound echo signals reflected from the target object to thereby form receive signals;
d3) applying delays to the receive signals to thereby form receive-focused signals; and
d4) forming three-dimensional ultrasound data corresponding to the plurality of ROIs based on the receive-focused signals.

12. The computer readable medium of claim 9, further comprising:
f) displaying the plurality of three-dimensional ultrasound images corresponding to the plurality of the ROIs.
